(19) United States
(12) Patent Application Publication
(10) Pub. No.: US 2026/0230562 A1
(43) Pub. Date: Aug. 6, 2026

(54) A Rescue Device and Method of Use Thereof

(71) Applicants: Renyu LIU, Media, PA (US); Anwen LIU, Media, PA (US)

(72) Inventors: Renyu LIU, Media, PA (US); Anwen LIU, Media, PA (US)

(21) Appl. No.: 19/159,757

(22) PCT Filed: Feb. 27, 2024
(86) PCT No.: PCT/US2024/017532

§ 371 Date: Aug. 26, 2025

Related U.S. Application Data
(60) Provisional application No. 63/487,415, filed on Feb. 28, 2023.

(51) Int. Cl. H04M 11/04; A61M 15/08; G16H 10/65; G16H 40/67

(52) U.S. Cl. CPC H04M 11/04; A61M 15/08; G16H 10/65; G16H 40/67; A61M 2205/59; A61M 2209/088

(57) ABSTRACT

A rescue device for rescuing a victim and method of use thereof. Such device comprises one portion/compartment for storing and administering a drug, e.g., naloxone, epinephrine, or any medications for treating a life-threatening situation, and an alert system, such as an alarm, that is triggered to an emergency call when the device is placed in a drug administration state by, for example, pulling the device out or opening the device.

### TECHNICAL FIELD

The present disclosure relates to a device system specifically for a drug delivery, preferably by an intranasal delivery, with an alarm system that can be triggered for an emergency call.

### BACKGROUND

There are many life-threatening situations that need medication(s) readily available for life threatening situations followed by emergency medical service (EMS) call (911 in the US, Canada etc.). For example, the data from the National Center for Health Statistics of the Centers for Disease Control and Prevention (CDC) estimated that there were 107,622 deaths from drug overdose in 2021 in the United States due to lack of immediate medication and EMS call. While the increase (15% from 2020 to 2021) is only 50% less than that from 2019 to 2020 (30% jump), it represents an additional 10,787 lives lost as compared to that in the previous year. The most worrisome finding is that the majority of the opioid overdose deaths (72%) happened in the young population (between 25 and 54 years old), who are supposed to be part of the work force of the society and key members of a family.

The current rescue approach for an opioid overdose is using a drug, naloxone. Dr. Jack Fishman and Dr. Mozes Lewenstein applied for the naloxone patent in 1961. The US Food and Drug Administration (FDA) approved naloxone as an opioid overdose treatment 10 years later in 1971. Naloxone is now routinely used by medical professionals to treat opioid overdose. Naloxone can be administered by various routes including intranasal, intravenous, and intramuscular. It has no side effects with a very high safety margin. The brand name of naloxone is well-known as “Narcan”. While there are many methods to deliver naloxone to rescue opioid overdose, intranasal naloxone administration remains the most convenient method to rescue opioid overdose effectively outside of hospital settings. On Jun. 21, 2022, the FDA approved the second generic naloxone intranasal spray to treat opioid overdose.¹

However, the availability of intranasal naloxone for immediate emergency administration is one of the biggest barriers of opioid overdose rescue. When someone has an opioid overdose, the victim or nearby persons may not have the intranasal naloxone immediately available. To develop a naloxone intranasal device that is easily wearable or carried that would encourage people to always carry the intranasal device is needed. Another major issue is that intranasal naloxone is not as effective as other more invasive administration routes like intramuscular and intravenous.² The half-life of naloxone is only about 60-90 minutes, and the half-life of many opioids are much longer than that of naloxone. The respiratory depression could potentially reoccur resulting in death. Naloxone administration could also trigger severe withdrawal syndrome which could potentially be life-threatening. Therefore, even after intranasal naloxone administration, a call to 911 is needed and the patient needs to be sent to a nearby hospital for additional monitoring and therapy. Thus, an alarm function of the naloxone rescue device should be added. This is true for many other life-threatening situations, like cardiac arrest, anaphylaxis, and stroke.

### SUMMARY

This present disclosure provides a rescue device with a combination of a drug delivery and alarm systems and method of use thereof. More specifically, the present disclosure provides a rescue device with intranasal drug delivery combined with an alarm and method of use thereof. The rescue device disclosed herein comprises an administering portion/compartment for storing and administering a drug, and an alert system. The method comprises using the rescue device to deliver the drug and to trigger emergency service call with the alarm to rescue a victim of an overdose of a drug or medication and/or under any life-threatening situation that requires an emergency service call, such as for making 911 calls in the US; 120 calls in China; 112 calls in European countries. The rescue device disclosed herein can also be used in other situations when only the intranasal drug delivery or only the alarm system of the device is needed (for example, when one is in a dangerous situation, the alarm can be trigged to alert nearby individuals to call EMS phone number).

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the drawings with the descriptions. The components in the drawings and descriptions are not necessarily to scale, emphasis instead being placed upon clearly illustrating the principles of the present disclosure. Moreover, in the drawings, like reference numerals designate corresponding parts throughout the several views.

FIGS. 1A and 1B show front perspective views of an example of the opioid overdose rescue device of the present disclosure in accordance with a representative embodiment in a non-administering state and in an administering state, respectively.

FIGS. 2A-2D show transparency front, side, top and perspective views, respectively, of the device shown in FIGS. 1A and 1B in the non-administering state shown in FIG. 1.

FIG. 3 shows a perspective transparency view of the rescue device shown in FIG. 2B coupled to a necklace.

FIG. 4 shows a rescue device in accordance with another exemplary embodiment in which the device has a mushroom shape.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

This present disclosure provides a rescue device with unique and novel features as summarized above in the summary and described below in detail. The present disclosure further provides a method of using the rescue device for rescuing victims of drug overdose (such as for opioid and/or other drug or medication overdose) and/or for rescuing people under any emergency situations.

To overcome the current opioid overdose rescue barriers, the present disclosure provides a rescue device that is easy to carry and/or fashionable (or cute) to wear. In certain embodiments, the rescue device comprises an administration portion/compartment for storing and administering a drug, such as administering naloxone, epinephrine, and/or other medications, preferably intranasally, for rescuing a drug overdose and/or for treating a life-threatening situation, and an alert system comprising an alarm portion/compartment comprising a battery chamber for a battery and an alarm to make a loud alarm sound with an announcement of something like “please call 911 for opioid overdose” (when such device is used in places where 911 is used as an EMS phone number; this will need to change to some other local EMS phone number in other localities) to trigger an EMS call with the alarm while administration of the drug, e.g., naloxone, epinephrine, or other medications and/or for treating life threatening situations, is carried out. The rescue device disclosed herein can also be used in other situations when other alarm system is not available or disabled.

In certain embodiments, the administration portion/compartment of the device can be attached to a keychain that may enable more people to carry the rescue device to make it readily available to rescue a victim, anywhere and anytime. In certain embodiments, the rescue device can be color coded to indicate that it is for medical usage. For instance, one embodiment of the rescue device provided herein can be colored in red, pink, and/or any other bright colors. In certain embodiments, the rescue device of the present disclosure can be made in various cute, fashionable, easy-to-carry and/or wearable shapes, such as cross shape, diamond shape, heart shape, star shape, etc. The rescue device of the present disclosure can be made by any suitable material that is wearable or carriable on, for example, a key chain.

In certain embodiments, the alert system further comprises an elastic metal component that contacts and triggers the alarm and EMS calls (For example, 911) and/or makes an EMS call (for example, “call 911!”) announcement. The alarm in the alarm portion/compartment preferably provides a high-pitched alarm sound and is designed and accompanied with an education campaign to the public. This is similar to an Amber alert campaign to alert the community that there is a victim, such an opioid overdose patient, nearby. The alert alarm may provide a very unique sound that can be recognized by people in the general public. The alarm preferably is triggered when the administration of a drug, e.g., naloxone, is being immediately needed/performed. In other embodiments, a button to mute the alarm may optionally be included. Furthermore, in certain embodiments, the alert system may comprise any suitable tracking system, e.g., GPS tracking system, iPhone, Apple watch. etc., and/or be connected/linked to any mobile device (e.g., cell phone, etc.) and/or databases related to the patient's location, medical, physical, healthcare records, and may be able to alert the patient's primary care physician and/or his/her affiliated hospitals/clinics.

In other embodiments, the rescue device may comprise a container for storing and administering a drug, such as naloxone, epinephrine, or other medications. The container can be attached to a wearable, carriable (attached to any of the body part), and may include a chain, such as a necklace, and/or be connected or hidden in a bra, for example. The container can be shaped in various fashionable and/or favorable cute shapes, such as mushroom shape, heart shape, diamond shape, star shape, etc., and can be color coded. In certain embodiments, the rescue device may not comprise an alarm and a battery in order to reduce cost and/or to make it easy to carry.

In certain embodiments, the rescue device disclosed herein can be used for rescuing opioid and/or other medications overdose victims. In certain embodiments, for opioid overdose, the rescue device disclosed herein contains naloxone that can be intranasally administered with the rescue device disclosed herein. In other embodiments for rescuing overdose of other drugs and/or medications, the rescue device disclosed herein contains medication(s), such as epinephrine, and/or other medications for treating life threatening situations that can be intranasally administered with the rescue device disclosed herein.

The present disclosure also discloses a method of rescuing a victim of a drug overdose and/or under emergency situations by using the rescue device of the present disclosure.

Many modifications to the embodiments disclosed herein and other embodiments not explicitly disclosed herein will come to mind to one skilled in the art to which the disclosed devices and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### DEFINITIONS

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,′ ”involved,“ and ”such as“ are used in their open, non-limiting sense and may be used interchangeably. Further, the term ”comprising“ is intended to include examples and aspects encompassed by the terms ”consisting essentially of“ and ”consisting of.′ Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a catalyst,” “a metal,” or “a substrate,” includes, but are not limited to, mixtures or combinations of two or more such catalysts, metals, or substrates, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

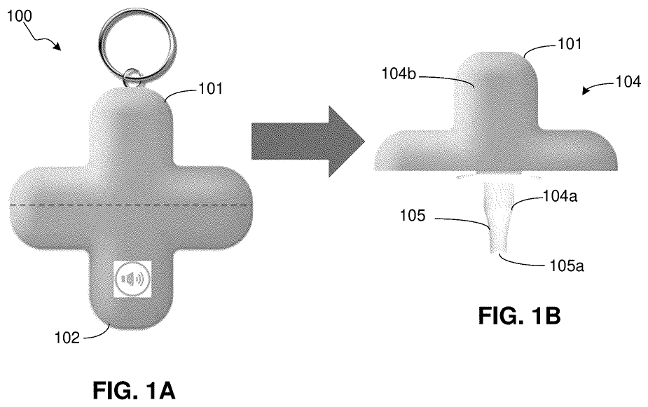

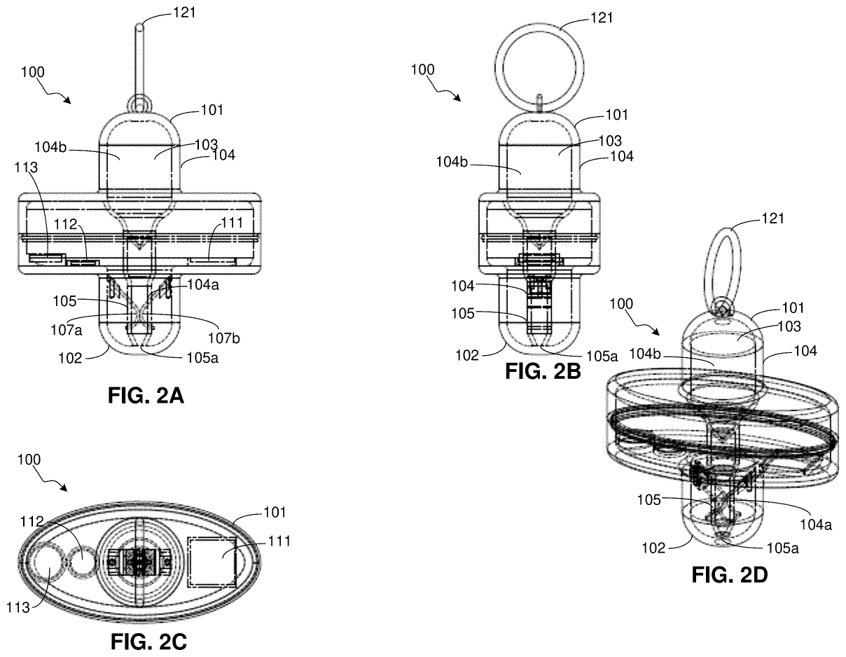

FIGS. 1A and 1B show front perspective views of the intranasal drug delivery rescue alarm device (for example: opioid overdose rescue alarm device) 100 (attached to a keychain for easy carry purpose) of the present disclosure in accordance with a representative embodiment in a non-administering state and in an administering state, respectively. FIGS. 2A-2D show transparency front, side, top and perspective views, respectively, of the device 100 in the non-administering state. The views of FIGS. 2A-2D are transparent to allow internal features of the device 100 to be seen. FIGS. 1A-2D illustrate a prototype of an embodiment of the rescue device 100 comprising a first portion 101 that acts as the administration portion 101 and a second portion 102 that acts as the case for the first portion 101. The first portion 101 comprises an intranasal administration portion having a reservoir and an applicator and the second portion 102 comprises a compartment for housing an alarm and a battery for powering the alarm.

In accordance with this exemplary embodiment, the alarm is triggered when the first portion 101 is pulled out of the case, i.e., separated from the second portion 102. As will be described below in more detail, in accordance with this exemplary embodiment, the second portion has two flexible metal arms 107a and 107b disposed therein that make electrical contact with one another when the first portion 101 is pulled out of the case (the second portion 102), thereby triggering the alarm and “call 911” announcement (or other EMS phone numbers in a specific country or region).

In this exemplary, or representative, embodiment, the device 100 has the shape of a cross, but as stated above, it could have any suitable shape. The device 100 preferably comprises first and second portions 101 and 102, respectively, that can couple with one another by any suitable coupling mechanism, such as a snap coupling arrangement or a tension coupling arrangement, for example. Preferably, the coupling mechanism is such that the portions 101 and 102 can be easily attached to, and detached from, one another.

The upper portion 101 comprises a compartment 103 (FIGS. 2A, 2B and 2D) that includes a reservoir for storing a drug to be administered to an overdose victim and an applicator 104 having a distal portion 104a that can be inserted into the nostril of the overdose victim and actuated by applying a squeezing, or pinching, force with a finger and thumb to a proximal portion 104b of the applicator 104. The proximal portion 104b comprises the reservoir for storing the drug and has a deflectable structure that is deflected by the force applied with the finger and thumb to push the drug from the reservoir into the distal portion 104a. The distal portion 104a has an elongated dispenser 105 that is configured, or adapted, to fit inside of a nostril of the overdose victim. When the person administrating the drug uses their finger and thumb to press inwardly on the proximal portion 104b of the applicator 104, some or all of the drug stored in the reservoir is forced out of a dispenser tip 105a of the elongated dispenser 105 into the overdose victim's nasal cavity.

In accordance with the exemplary embodiment shown in FIGS. 1A-2D , the second portion 102 of the overdose rescue device 100 includes inwardly-directed flexible metal arms 107a and 107b (FIG. 2A) that operate as springs and as electrical contacts. A proximal end of the arm 107b is in contact with an electrical contact 111 of the alarm and a distal end of the arm 107 comes into contact with a distal end of arm 107a when the first portion 101 is separated from the second portion 102. A proximal end of the arm 107a is in contact with a contact 112 for the battery 113.

The flexible arms 107a and 107b press inwardly against, and grip, outer surfaces of the elongated dispenser 105 when the device 100 is in the non-administering state shown in FIG. 1A. This spring force can be sufficiently strong that no other snap or tensioning mechanism is needed as the coupling mechanism for removably coupling the first and second portions 101 and 102, respectively, together. When the first and second portions 101 and 102, respectively, are coupled with one another to place the device 100 in the non-administering state, the arms 107a and 107b are pushed away from one another by the force exerted on them by the outer surfaces of the elongated dispenser 105. In this state, the arms 107a and 107b are biased inwardly against the outer surfaces of the elongated dispenser 105 and do not make electrical contact with one another.

When the first and second portions 101 and 102, respectively, are separated from one another to place the device 100 in the administering state, the distal ends of the arms 107a and 107b come into contact with one another due to the spring forces of the arms 107a and 107b. In this state, the arms 107a and 107b provide an electrically-conductive path for electricity to flow from the battery 113 to the alarm 111, thereby triggering the alarm 111 with an emergency voice announcement of “please call 911 (or 120, 112 etc. the dedicated EMS number in each country or region), someone needs to be rescued now” or something alike.

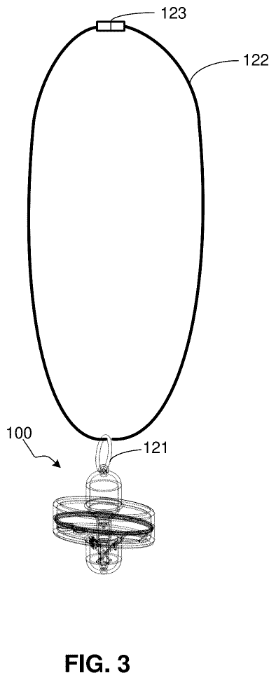

FIG. 3 is a perspective transparency view of the rescue device 100 shown in FIG. 2B coupled to a necklace 122. A chain or strand of the necklace 122 passes through a ring (or keychain) 121 of the rescue device 100 and has a clasp (or any of necklace) 123 that can be detached to allow a different necklace or chain (e.g., a key chain) to be attached to the rescue device 100. The purpose of this illustration is to demonstrate that this device is designed to be attached to anything for easy carrying purposes for the items that a person will carry on daily basis to increase the availability of the device and medication when needed any time and any places.

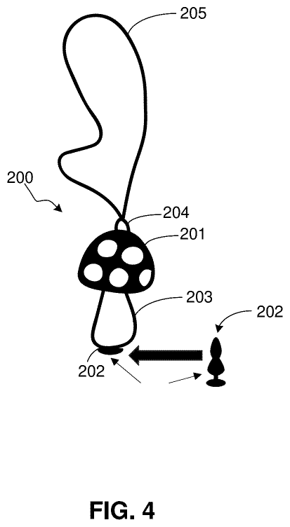

FIG. 4 shows a rescue device 200 in accordance with another exemplary embodiment in which the device 200 has a mushroom shape. A medication chamber 201 of the device 200 has a cap or a screw/plug 202 or a spray nose hole for the opening or delivery of the medication of the rescue device 200 to encase the dispensing portion 203 in the second portion of the rescue device. The first portion 201 includes the reservoir for storing the drug or medication to be administered intranasally to rescue an overdose victim or anyone that needs an immediate rescue medication. The device 200 includes a ring or keychain 204 through which a necklace or chain (or other convenient carrying device) 205 can be passed.

It should be emphasized that the described embodiments of the present disclosure are merely possible examples of implementations set forth for a clear understanding of the principles of the disclosure. The shape and color can be anything for decorative, stylish, or personal preference purposes. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1.FDA(USA)
   https://www.accessdata.fda.gov/scripts/cder/ob/results_product.cfm?Appl_Type=A&App I_No=211951.
2. Dietze, P.; Jauncey, M.; Salmon, A.; Mohebbi, M.; Latimer, J.; van Beek, I.; McGrath, C.; Kerr, D., Effect of Intranasal vs Intramuscular Naloxone on Opioid Overdose: A Randomized Clinical Trial. JAMA Netw Open 2019, 2 (11), e1914977.
3. https://youtu.be/ML9S24q8J6U

1. A rescue device comprising an administering portion/compartment for storing and administering a drug, and an alert system.

2. The rescue device of claim 1, wherein the alert system comprises an alarm.

3. The rescue device of claim 2, wherein the alert system further comprises a battery chamber for a battery.

4. The rescue device of claim 2, wherein the alert system further comprises elastic metal component(s) that contact and trigger the alarm and call 911 announcement.

5. The rescue device of claim 2, wherein the alert system further comprises a button to turn on or off/mute of the alarm.

6. The rescue device of claim 1, wherein the alert system comprises a suitable tracking system connected or linked to a patient's location, medical or healthcare record, and/or patient's physician or affiliated hospital or clinics.

7. The rescue device of claim 1, wherein the device is in a cross shape and color coded in red.

8. The rescue device of claim 1, wherein the device is in a heart, star, diamond, or the like shape with or without color coded.

9. The rescue device of claim 1, wherein the device is attached to a keychain and/or a necklace.

10. (canceled)

11. The rescue device of claim 1, wherein the device is shaped in mushroom shape, or any other suitable shape.

12. The rescue device of claim 1, wherein the device is made by any suitable material that is wearable.

13. The rescue device of claim 1, wherein the drug is naloxone.

14. The rescue device of claim 13, wherein naloxone is administered intranasally.

15. A method of rescuing a victim of an overdose of a drug or medication using the rescue device of claim 1.

16. The method of claim 15, wherein an alert system is trigged when the administration of a drug stored in the rescue device is pulled out.

17. The method of claim 16, wherein the alert system comprises an alarm that calls 911 announcement.

18. The method of claim 16, wherein the alert system comprises a suitable tracking system that connects or links to a patient's location, medical or healthcare record, and/or patient's physician or affiliated hospital or clinics.

19. The method of claim 16, wherein drug or medication is stored in the rescue device and used to rescue a victim of overdose of certain drugs or medications for life threatening situations.

20. The method of claim 19, wherein drug or medication is administered intranasally.

21. The method of claim 20, wherein the drug or medication is naloxone or epinephrine or any medication for life threatening situations.
